(19) United States
(12) Patent Application Publication
(10) Pub. No.: US 2002/0129407 A1
(43) Pub. Date: Sep. 12, 2002

(54) Plant gene required for male meiosis

(76) Inventor: Hong Ma, State College, PA (US)

Correspondence Address: Janet E. Reed, WOODCOCK WASHBURN LLP, One Liberty Place - 46th Floor, Philadelphia, PA, 19103, US

(21) Appl. No.: 09/821,839

(22) Filed: Mar. 29, 2001

Related U.S. Application Data
(60) Provisional application No. 60/193,523, filed on Mar. 31, 2000.

(51) Int. Cl. A01H 5/00; C12N 15/00; C12N 15/09; C12N 15/29; C07K 14/00; C07K 16/00; C12N 5/04

(52) U.S. Cl. 800/298; 536/23.6; 536/24.33; 530/350; 530/387.1; 435/320.1; 435/419; 536/24.1

(57) ABSTRACT

Provided in the present invention is a novel gene, SDS, the disruption of which is associated with abnormal homolog attachment during meiosis in plants. Also provided are transgenic plants and mutants of the SDS gene as well as a polypeptide encoded by the SDS gene.

This application claims the benefit of priority to U.S. Provisional Application 60/193,523, filed on Mar. 31, 2000, the entirety of which is incorporated by reference herein.

Pursuant to 35 U.S.C. §202(c), it is acknowledged that the U.S. Government has certain rights in the invention described herein, which was made in part with funds from the National Science Foundation No. MCB-9896340.

### FIELD OF THE INVENTION

This invention relates to the field of plant breeding and reproduction. In particular, this invention relates to a novel gene involved in regulating and controlling meiosis and cell division.

### BACKGROUND OF THE INVENTION

Various scientific and scholarly articles are referred to throughout the specification. These articles are incorporated by reference herein to describe the state of the art to which this invention pertains.

Meiosis is essential for eukaryotic sexual reproduction, halving the number of chromosomes into four products. This halving requires that homologous chromosomes (homologs) interact properly during prophase I and remain attached at metaphase I. Classical cytology in plants and animals, and molecular genetics and molecular cytology in yeast, Drosophila, C. elegans, and other organisms, has led to the current understanding of the interaction between homologs during prophase I. In particular, it is universal that homologs are attached until the onset of anaphse I. The maintenance of homolog attachment is thought to require both meiotic recombination and sister chromatid cohesion. In yeast, the sister-chromatid cohesin is required for normal meiosis I, and the removal of cohesin is necessary for homolog and sister chromatid separation at meiosis I and II, respectively. In recent years, several Arabidopsis mutations affecting meiosis have been isolated. However, all of these reported mutants are still fertile enough to be maintained as homozygotes.

Many questions remain about the regulation of homolog attachment. In particular, the manner in which homolog attachment is regulated by cell cycle regulators is poorly understood. Accordingly, there is a need in the art for isolation and characterization of plant genes involved in meiosis. Such genes would provide utility in the manipulation and regulation of plant fertility. In addition, there is a need for transformed plants that fail to produce pollen and/or are sterile.

### SUMMARY OF THE INVENTION

Provided in the present invention is a novel gene (referred herein as SDS), which is associated with regulation of meiosis in plants. The invention further provides transgenic plants and mutants that exhibit abnormal homolog interaction during meiosis. The SDS gene or its corresponding protein has not previously been described in plants.

According to one aspect of the present invention, a novel gene, SDS, is provided. The SDS gene is located on Arabidopsis thaliana chromosome 1. The SDS genes on the BAC clone designated as F10B6 maps to about 23.6 centimorgan from the left (top) end of chromosome 1, and is flanked by the BAC clones T5E21 (left) and T15D22. These BACs are further flanked by markers g2358 and SGCSNP303 on the left and SRP54A on the right sides. The genomic sequence of the Arabidopsis SDS gene is set forth in SEQ ID NO:4.

The disruption of the SDS gene is associated with a failure to maintain homolog attachment during meiosis. In a preferred embodiment, this gene encodes a protein with a cyclin or cyclin-like domain. In a more preferred embodiment, the gene contains exons that encode a protein that is 500-600 amino acids in length, preferably approximately 578 amino acids in length. In a yet more preferred embodiment, the nucleic acid molecule contains an open reading frame that encodes a protein that is at least 50% identical over its full length to SEQ ID NO:2, and in a particularly preferred embodiment encodes SEQ ID NO:2. In a more particularly preferred embodiment, the nucleic acid molecule is comprised of SEQ ID NO:1. Provided with this aspect of the invention is a cDNA molecule comprising the exons of the gene which encode a polypeptide 500-600 amino acids in length, more preferably about 578 amino acids in length. Also provided is a nucleic acid has a sequence that is selected from SEQ ID NO:1, a nucleic acid encoding a sequence that is at least 70% identical to SEQ ID NO:1. Provided with this aspect of the invention is a polypeptide that is produced by the expression of the isolated nucleic acid molecule, and antibodies immunolgically specific for the polypeptide. Also provided with this aspect of the invention is a nucleic acid molecule of at least 15 nucleotides in length, preferably at least 20 nucleotides in length, and most preferably at least 30 nucleotides in length, that is identical in sequence to a portion of the SDS gene located on Arabidopsis thaliana chromosome 1. In a preferred embodiment, the invention provides a nucleic acid molecule of at least 15, preferably 20, and most preferably 30 nucleotides in length, that is identical to or complementary to a consecutive 15, 20 or 30 nucleotide portion, respectively, of the sequence set forth in SEQ ID NO:1.

Also provided in the present invention are recombinant materials and methods for the production of the SDS gene and sds mutants. In another aspect, the invention relates to methods for using such polypeptides and polynucleotides. In a further aspect, the invention relates to methods for identifying agonists and antagonists or inhibitors using the materials provided by the invention. In a still further aspect, the invention relates to assays for detecting abnormalties in plants associated with inappropriate homolog attachment during meiosis.

Also provided in the present invention are mutant plants and transgenic plants that overexpress or underexpress SDS. Portions of such plants, cells of such plants, and reproductive units (e.g., seeds) of such plants are also contemplated in the present invention.

Other features and advantages of the present invention will be better understood by reference to the drawings, detailed description and examples that follow.

### BRIEF DESCRIPTION OF THE DRAWINGS

FIG. 1 shows the SDS amino acid sequence of SEQ ID NO:2. The cyclin domain is denoted by underlining.

FIG. 2 shows the alignment of the cyclin homolog (cyclin box) of SDS (SEQ ID NO:5) with those of Arabidopsis cyclins 2b (SEQ ID NO:6) and 2a (SEQ ID NO:7). In the consensus sequence, a plus sign indicates conservative substitutions.

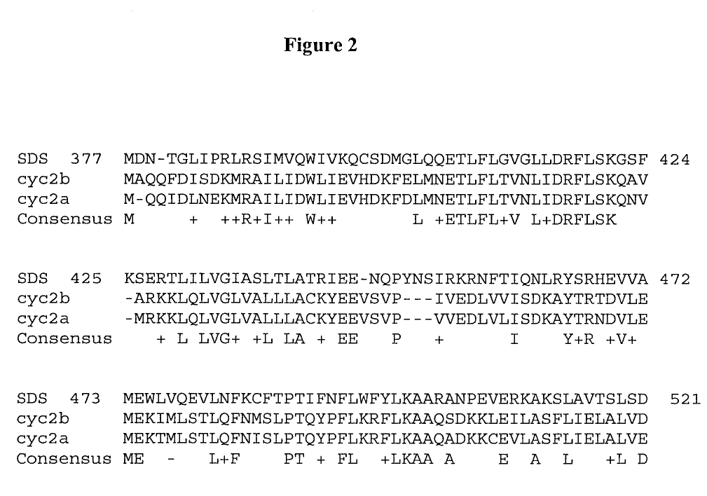

FIG. 3 is a chart showing the identity and similarity among SDS and four Arabidopsis cyclins: 2b, 2a, 3b and D. Similarities between SDS and cyc2a or cyc2b are higher than that between any of these three and cycD.

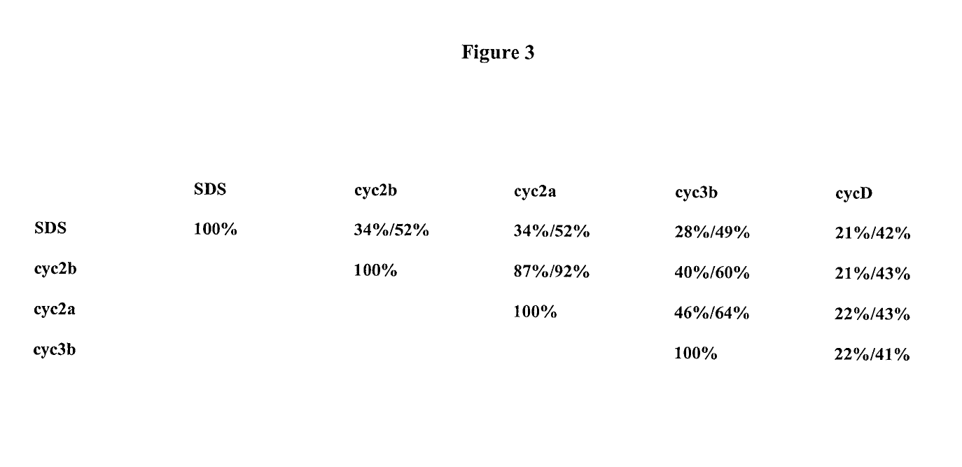

### DETAILED DESCRIPTION OF THE INVENTION

I. Definitions

Various terms relating to the biological molecules of the present invention are used hereinabove and also throughout the specifications and claims.

With respect to the genotypes of the invention, the terms “SDS” and “sds” are used. The term “SDS” is used to designate the naturally-occurring or wild-type genotype. This genotype has the phenotype of normal meiosis and normal microspores. The genotype of the mutant is abnormal homolog attachment during meiosis, and abnormally sized microspores. The term “sds” refers to a genotype having recessive mutation(s) in the wild-type SDS gene. The phenotype of sds individuals is abnormal meiosis during prophase I and metaphase. Where used hereinabove and throughout the specifications and claims, the term “SDS” refers to the protein product of the SDS gene.

In reference to the mutant plants of the invention, the term “null mutant” is used to designate an organism or genomic DNA sequence with a mutation that causes the product of the SDS gene to be non-functional or largely absent. Such mutations may occur in the coding and/or regulatory regions of the SDS gene, and may be changes of individual residues, or insertions or deletions of regions of nucleic acids. Such mutations may also occur in the coding and/or regulatory regions of other genes which may regulate or control the SDS gene and/or the product of the SDS gene so as to cause said gene product to be non-functional or largely absent.

With reference to nucleic acids of the invention, the term “isolated nucleic acid” or “polynucleotide” is sometimes used. These terms, when applied to genomic DNA, refers to a DNA molecule that is separated from sequences with which it is immediately contiguous (in the 5′ and 3′ directions) in the naturally-occurring genome of the organism from which it was derived. For example, the “isolated nucleic acid” may comprise a DNA molecule inserted into a vector, such as a plasmid or virus vector, or integrated into the genomic DNA of a procaryote or eukaryote. An “isolated nucleic acid molecule” may also comprise a cDNA molecule or a synthetic DNA molecule.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form .

Nucleic acid sequences and amino acid sequences can be compared using computer programs that align the similar sequences of the nucleic or amino acids thus define the differences. For purposes of this invention, the DNAStar program (DNAStar, Inc., Madison, Wis.) and the default parameters used by that program are the parameters intended to be used herein to compare sequence identity and similarity. Alternately, the Blastn and Blastp 2.0 programs provided by the National Center for Biotechnology Information (at www.ncbi.nlm.nih.gov/blast/; Altschul et al., 1990, J. Mol. Biol. 215:403-410) using a gapped alignment with default parameters, may be used to determine the level of identity and similarity between nucleic acid sequences and amino acid sequences.

The term “substantially the same” refers to nucleic acid or amino acid sequences having sequence variation that do not materially affect the nature of the protein (i.e. the structure, thermostability characteristics and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides by a sequence analysis program.

“Identity” and “similarity” can be readily calculated by known methods. Nucleic acid sequences and amino acid sequences can be compared using computer programs that align the similar sequences of the nucleic or amino acids thus define the differences. In preferred methodologies, the BLAST programs (NCBI) and parameters used therein are employed, and the DNAstar system (Madison, Wis.) is used to align sequence fragments of genomic DNA sequences. However, equivalent alignments and similarity/identity assessments can be obtained through the use of any standard alignment software. For instance, the GCG Wisconsin Package version 9.1, available from the Genetics Computer Group in Madison, Wis., and the default parameters used (gap creation penalty=12, gap extension penalty=4) by that program may also be used to compare sequence identity and similarity.

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

With respect to antibodies of the invention, the terms “immunologically specific” or “specific” refer to antibodies that bind to one or more epitopes of a protein of interest, but which do not substantially recognize and bind other molecules in a sample containing a mixed population of antigenic biological molecules.

With respect to oligonucleotides, but not limited thereto, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

A “vector” is a replicon, such as plasmid, phage, cosmid, or virus to which another nucleic acid segment may be operably inserted so as to bring about the replication or expression of the segment.

The term “reporter gene” refers to genetic sequences which may be operably linked to a promoter region forming a transgene, such that expression of the reporter gene coding region is regulated by the promoter and expression of the transgene is readily assayed.

The term “selectable marker gene” refers to a gene product which when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

The term “DNA construct” refers to genetic sequences used to transform plants or other organisms (e.g., bacteria, yeast). When transforming plants, these constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 2001.

II. Description

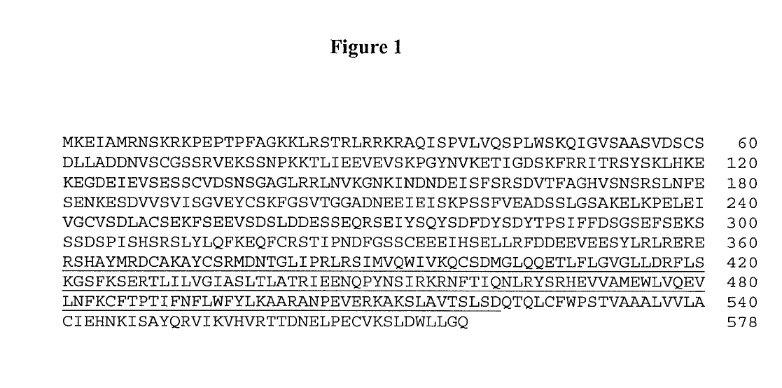

In accordance with the present invention, a gene is provided that is a novel regulator of meiosis. This gene, SDS, was initially isolated from Arabidopsis thaliana. The sds mutant has a defect in maintaining homolog attachment at late prophase I, and is caused by a Ds transposon insertion. Sequence analysis indicates that the SDS gene encodes a protein with strong similarity to known cyclins. The cyclin-like domain of SDS (as underlined in FIG. 1) has about 28-34% amino acid sequence identity to plant A and B type cyclins, and 21% identity to an Arabidopsis D type cyclin. These levels of similarity are similar to those found between different types of cyclins.

Cyclins and CDKs (cyclin-dependent kinases) are central regulators of the mitotic cell cycle, and regulate mitotic sister chromatid separation at anaphase. Furthermore, there is evidence that they also regulate meiosis including meiotic sister chromatid cohesion. It is likely that cyclin and CDK are critical regulators of homolog attachment in meiosis I. The SDS gene encodes a meiosis-specific cyclin that activates a cyclin-dependent kinase (CDK) to regulate the activities of other proteins that maintain homolog attachment. Furthermore, there is evidence that SDS interacts with the ASK1 and SYN1 genes, which are also involved in the regulation of meiosis.

SDS is not expressed during vegetative development, late flower development, nor in fruit and seed development. In situ hybridization indicates that expression in the anther is restricted to the meiotic cells. Therefore, SDS appears to be a meiosis-specific gene, encoding a novel type of cyclin.

The abnormal homolog attatchment of the sds mutant in male meiosis is a recessive trait. This is evidenced by the genetic characterization performed on plants having the sds mutation. One Ds insertional line (F2) segregated for sterile plants at the F3 generation with a frequency of roughly one quarter mutants, suggesting that the F2 plant was heterozygous for a recessive mutation. In addition, the progeny of a cross by pollinating the mutant pistil with normal pollen were normal, confirming that the mutation was indeed recessive.

Further, phenotypic characterization also supports that the sds mutant of the present invention is defective in male meiosis. The sds mutant is normal in vegetative and flower organ development. Wild-type Arabidopsis produces normal pollen grains. However, the sds mutant produced a reduced number of abnormal pollen grains with variable sizes. Analysis of immature anthers showed that the mutant microspores also had different sizes, in contrast to the normal microspores. Further examination revealed that whereas a normal meiosis produces four microspores of equal size in a tetrad, the mutant produces “tetrads” with four to six, or eight, microspores that had variable sizes. As pollen is allergenic to humans and other organisms, plants mutant in SDS and therefore having a phenotype of defective pollen and male sterility could be extremely beneficial to the public.

As described above, cells defective in the SDS gene exhibit abnormal homolog interaction. Several hundred wild-type and mutant meiotic cells were analyzed in the present invention using a chromosome spread and DAPI staining procedure. During leptotene, chromosomes began to condense and form visible thin lines. At late zygotene or early pachytene, homologs had initiated synapsis, which was completed at pachytene. In diplotene, the homologs partially separated, but associated at the chiasmata. At diakinesis, chromosomes had further condensed, and were easily visible as the five bivalents. In sds mutant plants, during male meiotic prophase I, leptotene, late zygotene or early pachytene, and pachytene appeared normal with condensed and attached homologs. However, by the time of diakinesis in sds mutant cells, the homologs were not properly attached. Ten unattached chromosomes could easily be visualized using microscopy well known to those of skill in the art.

In the wild-type at metaphase I, the five bivalents aligned at the equatorial plane; however, the sds mutant univalents failed to align properly. The mutant anaphase I and telophase I were difficult to recognize, in part due to the abnormal distribution of chromosomes. However, the phases could be assigned based on available clues. Because the homologs had prematurely separated, anaphase I was abnormal and single chromosomes were scattered and some were elongated, presumably due to stretching by the anaphase I spindle.

In the wild-type at diakinesis, the bivalents had “X” or “Y” shapes, presumably representing homologs connected via chiasmata. In the sds mutant, because homologs did not remain attached at diakinesis, there should not be any chiasma. However, the single chromosomes, though seen as smaller entities than those or the bivalents, appeared to have “X” or “Y” shapes as well. This feature was not seen in mutants other than sds that have unattached homologs. Therefore this did not seem to be a common feature in unattached chromosomes at diakinesis. These images may represent chromosomes that have partially separated sister chromatids, mostly likely along the arms but not at the centromere.

During meiosis II in the wild-type cells, the two groups of chromosomes were separated by a band of organelles. Chromosomes first condensed during prophase II and were highly condensed at metaphase II. Sister chromatids separated at anaphase II, moved to opposite poles and decondensed at telophase II and decondensed to form four nuclei. In the sds mutant, the distribution of chromosomes were abnormal due to a defective meiosis I, but the behavior of chromosomes appeared normal. Chromosomes condensed at prophase II, became highly condensed at metaphase II. More than two clusters were often observed, likely due to the scattering during meiosis I.

Sister chromatids separated at anaphase II, and moved to opposite poles at telophase II. Sister chromatid separation in meiosis II was generally normal in sds mutant cells. This suggests that meiosis II spindles can form around each cluster of chromosomes, even a single chromosome. This was further supported by an analysis of spindle structure. The cells formed “tetrads” containing 6 or 8 spores. These are the two most frequent classes of tetrads. Occasionally, an odd number of spores were found in a tetrad, suggesting that the separation of sister chromatids is sometimes not normal.

Immunofluorescence microscopy was performed to examine the meiotic spindle structure in wild-type and sds cells. Wild-type metaphase I cells possessed spindles. Because the cells were still intact and the chromosomes were not spread, the metaphase I chromosomes appeared as a cluster. At late anaphase I or early telophase I, there were two clusters of chromosomes, representing the separated homologs. At late telophase, a new microtubule structure was formed that was much broader than the spindle. This structure was similar to the plant microtubule structure called phragmoplast formed at mitotic telophase. Phragmoplast is thought to be important for the positioning of the new cell plate separating the two newly formed nuclei. In meiosis I, this microtubule structure has no specific name, and its position coincides with the position of a new organellar band. This organellar band was also present in sds cells.

In sds mutant cells, the metaphase I spindles exhibited a morphology that was more complex than that of the wild-type spindle, and the chromosomes were more scattered. The shape of the mutant spindle could be interpreted as a composite spindle of several smaller spindles, each centered around a single chromosome or a cluster of chromosomes. In other cells, more scattered chromosomes were observed. These results are consistent with the idea that in plant cells chromosomes play a critical role in organizing spindles. Therefore, the spindle morphology in the sds mutant is due to abnormal distribution of chromosomes, although a regulation of spindle structure by SDS cannot be ruled out. Microtubule structures in mutant cells were broad, suggesting that the stage is similar to telophase I. This also suggests that the microtubule structure at this stage does not depend on proper distribution of chromosomes.

We have previously grown the sds mutant under constant light, and did not observe any seed production. In a greenhouse it was observed that some seedpods in sds were slightly enlarged and had one or two seeds, producing 50-100 seeds per plant. Some of the seeds from sds mutant plants were planted, about ⅓ to ½ of seedling exhibited morphological abnormalities at the seedling or vegetative stages, consistent with the idea that they might be aneuploid due to unequal chromosome distribution. Those sds progeny plants that reached maturity all had the same very low fertility phenotypes of the sds mutant, indicating that they are not from cross pollination. When grown under constant light, these plants were again sterile. Plants defective in SDS were grown under better controlled short day conditions and they also produced some seeds. Therefore, under some conditions, such as short days, the sds plants occasionally produce some viable pollen.

When sds is used as the female in a cross with the wild-type, seeds are present, but in reduced numbers compared to a wild type plant. This suggests that sds has a reduced female fertility. It is possible that female meiosis is also affected in the sds mutant.

The novel features of the wildtype SDS and encoded protein as well as the mutant sds gene having been described above and in greater detail in the examples, the invention will now be described in detail. Thus, in a first aspect, the present invention relates to SDS peptides. Such peptides include isolated polypeptides comprising an amino acid sequence which has at least 70% identity, preferably at least 80% identity, more preferably at least 90% identity, yet more preferably at least 95% identity, most preferably at least 97-99% identity, to a region comprising at least 100 contiguous amino acids of that of SEQ ID NO:2 over the entire length of SEQ ID NO:2. In more preferred embodiments, percent identity is compared to a region comprising at least 150 contiguous amino acids of SEQ ID NO:2, with 200 contiguous amino acids being even more preferred and 250 continguous amino acids being the most preferred. In preferred embodiments, such peptides share the recited percent identity over the cyclin domain of SEQ ID NO:2, as denoted in FIG. 1. Such polypeptides also include those comprising the amino acid of SEQ ID NO:2.

Further peptides of the present invention include isolated polypeptides in which the amino acid sequence has at least 50%, 60% or 70% identity, preferably at least 80% identity, more preferably at least 90% identity, yet more preferably at least 95% identity, most preferably at least 97-99% identity, to the amino acid sequence of SEQ ID NO:2 over the entire length of SEQ ID NO:2. Such polypeptides include the polypeptide of SEQ ID NO:2. Further peptides of the present invention include isolated polypeptides encoded by a polynucleotide comprising the sequence contained in SEQ ID NO: 1 or the coding region of the sequence contained in SEQ ID NO:4.

In a further aspect, the present invention relates to SDS polynucleotides. Such polynucleotides include isolated polynucleotides comprising a nucleotide sequence encoding a polypeptide which has at least 50%, 60%, or 70% identity, preferably at least 80% identity, more preferably at least 90% identity, yet more preferably at least 95% identity, to the amino acid sequence of SEQ ID NO:2, over the entire length of SEQ ID NO:2. In this regard, polypeptides which have at least 97% identity are highly preferred, while those with at least 98-99% identity are more highly preferred, and those with at least 99% identity are most highly preferred. Such polynucleotides include a polynucleotide comprising the nucleotide sequence contained in SEQ ID NO:1 encoding the polypeptide of SEQ ID NO:2.

Also included in the present invention are polynucleotides that are at least 100 nucleotides in length, preferably at least 300 nucleotides in length, more preferably at least 500 nucleotides in length, most preferably at least 600 nucleotides in length. These polynucleotides have at least 70% identity to the cyclin domain of SEQ ID NO:1, with 80% identity being more preferred, 90% identity being highly preferred, 95% identity being still more highly preferred, and 97-99% identity being the most preferred. In one embodiment, percent identity is measured over the C-terminal 50% of the sequence set forth in SEQ ID NO:1.

Further polynucleotides of the present invention include isolated polynucleotides comprising a nucleotide sequence that has at least 50%, 60%, or 70% identity, preferably at least 80% identity, more preferably at least 90% identity, yet more preferably at least 95% identity, to a nucleotide sequence encoding a polypeptide of SEQ ID NO:2, over the entire coding region. In this regard, polynucleotides which have at least 97% identity are highly preferred, whilst those with at least 98-99% identity are more highly preferred, and those with at least 99% identity are most highly preferred.

Further polynucleotides of the present invention include isolated polynucleotides comprising a nucleotide sequence which has at least 50%, 60%, or 70% identity, preferably at least 80% identity, more preferably at least 90% identity, yet more preferably at least 95% identity, to SEQ ID NO:1 or SEQ ID NO:4, over the entire length of SEQ ID NO:1 or SEQ ID NO:4, respectively. In this regard, polynucleotides which have at least 97% identity are highly preferred, while those with at least 98-99% identity are more highly preferred, and those with at least 99% identity are most highly preferred. Such polynucleotides include a polynucleotide comprising the polynucleotide of SEQ ID NO:1 or SEQ ID NO:4 as well as the polynucleotides of SEQ ID NO:1 and SEQ ID NO:4.

The invention also provides polynucleotides that are complementary to all the above described polynucleotides.

The nucleotide sequence encoding the polypeptide of SEQ ID NO:2 may be identical to the polypeptide encoding sequence contained in SEQ ID NO:1 or it may be a sequence other than the one contained in SEQ ID NO:1, which, as a result of the redundancy (degeneracy) of the genetic code, also encodes the polypeptide of SEQ ID NO:2. The polypeptide of SEQ ID NO:2 is structurally related to other proteins of the cyclin family, having homology and/or structural similarity with cyclin.

Although the SDS genomic clone from Arabidopsis thaliana is described and exemplified herein, this invention is intended to encompass nucleic acid sequences and proteins from other plants that are sufficiently similar to be used instead of the Arabidopsis SDS nucleic acid and proteins for the purposes described below. These include, but are not limited to, allelic variants and natural mutants of SEQ ID NO:1, which are likely to be found in different species of plants or varieties of Arabidopsis. Because of the natural sequence variation likely to exist among SDS genes, one skilled in the art would expect to find up to about 20-30% nucleotide sequence variation, while still maintaining the unique properties of the SDS gene and encoded polypeptide of the present invention. Such an expectation is due in part to the degeneracy of the genetic code, as well as to the known evolutionary success of conservative amino acid sequence variations, which do not appreciably alter the nature of the encoded protein. Accordingly, such variants are considered substantially the same as one another and are included within the scope of the present invention.

Sds mutant plants are also part of the present invention. Mutants of Arabidopsis exhibit an abnormal meiosis with characteristics of meiotic regulation that have not been previously described. Such mutants are unable to maintain homolog attachment during late prophase I of male meiosis. The sds mutant of Arabidopsis was first isolated among Ds transposable lines due to visibly detectable fertility defect. This was further confirmed by microscopic examination.

It is also contemplated that the present invention encompasses not only other plant homologs of the SDS gene, but also using these homologs to better understand meiosis in other species.

Polynucleotides which are identical or sufficiently identical to a nucleotide sequence contained in SEQ ID NO:1, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than Arabidopsis) that have a high sequence similarity to SEQ ID NO:1. Typically these nucleotide sequences are 70% identical, preferably 80% identical, more preferably 90% identical, most preferably 95% identical to that of the referent The probes or primers will generally comprise at least 15 nucleotides, preferably, at least 30 nucleotides and may have at least 50 nucleotides. Particularly preferred probes will have between 30 and 50 nucleotides.

The following sections set forth the general procedures involved in practicing the present invention. To the extent that specific materials are mentioned, it is merely for purposes of illustration and is not intended to limit the invention. Unless otherwise specified, general cloning procedures, such as those set forth in Sambrook et al., Molecular Cloning, Cold Spring Harbor Laboratory (1989) (hereinafter “Sambrook et al.”) or Ausubel et al (eds) Current Protocols in Molecular Biology, John Wiley & Sons (2001) (hereinafter “Ausubel et al.”) are used.

III. Preparation of Sds Mutants, SDS Nucleic Acids, Proteins, Antibodies, and Transgenic Plants.

A. Isolation of sds Genetic mutants

Populations of plant mutants are available from which sds mutants in other plant species can be isolated. Many of these populations are very likely to contain plants with null mutations in the SDS gene. Such populations can be made by chemical mutagenesis, radiation mutagenesis, and transposon or T-DNA insertions. The methods to make mutant populations are well known in the art.

The nucleic acids of the invention can be used to isolate sds mutants in other species. In species such as maize where transposon insertion lines are available, oligonucleotide primers can be designed to screen lines for insertions in the SDS gene. Plants with transposon or T-DNA insertions in the SDS gene are very likely to have lost the function of the gene product. Through breeding, a plant may then be developed that is homozygous for the non-functional copy of the SDS gene. In order to maintain that plant, a heterozygous line is necessary that is a carrier for the instant mutation. The PCR primers for this purpose are designed so that a large portion of the coding sequence of the SDS gene are specifically amplified using the sequence of the SDS gene from the species to be probed (see Baumann et al., 1998, Theor. Appl. Genet. 97:729-734).

Other sds-like mutants can easily be isolated from mutant populations using the distinctive phenotype characterized in accordance with the present invention. This approach is particularly appropriate in plants with low ploidy numbers where the phenotype of a recessive mutation is more easily detected. Plants would then be screened for phenotype of the sds mutant: a reduced number of abnormal pollen grains with variable sizes, microspores having different sizes in the anther, and “tetrads” with four to six, or eight, microspores having variable sizes. That the phenotype is caused by an sds mutation is then established by molecular means well known in the art. Species contemplated to be screened with this approach include but are not limited to: aster, barley, begonia, beet, cantaloupe, carrot, chrysanthemum, clover, corn, cucumber, delphinium, grape, lawn and turf grasses, lettuce, pea, peppermint, rice, rutabaga, sugar beet, tomatillo, tomato, turnip, wheat, zinnia, cabbage, cauliflower, broccoli, brussel sprouts, chinese cabbage, canola, apple, peach, pear, alfalfa, soybean, sunflower and sorghum.

B. Isolation of SDS Genes

A gene can be defined by its mapped position in the plant genome. Although the chromosomal position of the gene can change dramatically, the position of the gene in relation to its neighbor genes is often highly conserved (Lagercrantz et al., 1996, Plant J. 9:13-20). This conserved micro-colinearity can be used to isolate the SDS gene from distantly related plant species. These genes and markers can be used to isolate the SDS gene in their midst, or to confirm the identity of an isolated SDS nucleic acid (described below). For example, the various coding sequences can be used to design probes to isolate the SDS gene on BAC clones or to map the chromosomal location of the SDS gene using recombination frequencies. Additionally, genes highly homologous to those on Arabidopsis BAC are already known in other species, and these homologous genes may be used to locate SDS in these genomes. There are several versions of these procedures, and all will be well known to those skilled in the art.

C. Isolation of SDS Nucleic Acid Molecules

Nucleic acid molecules encoding the SDS protein may be isolated from Arabidopsis or any other plant of interest using methods well known in the art. Nucleic acid molecules from Arabidopsis may be isolated by screening Arabidopsis cDNA or genomic libraries with oligonucleotides designed to match the Arabidopsis nucleic acid sequence of the SDS gene (SEQ ID NO:1). In order to isolate the SDS-encoding nucleic acids from plants other than Arabidopsis, oligonucleotides designed to match the nucleic acids encoding the Arabidopsis SDS protein may be likewise used with cDNA or genomic libraries from the desired species. If the SDS gene from a species is desired, the genomic library is screened. Alternatively, if the protein coding sequence is of particular interest, the cDNA library is screened. In positions of degeneracy, where more than one nucleic acid residue could be used to encode the appropriate amino acid residue, all the appropriate nucleic acid residues may be incorporated to create a mixed oligonucleotide population, or a neutral base such as inosine may be used. The strategy of oligonucleotide design is well known in the art (see also Sambrook et al.).

Alternatively, PCR (polymerase chain reaction) primers may be designed by the above method to encode a portion of the Arabidopsis SDS protein, and these primers used to amplify nucleic acids from isolated cDNA or genomic DNA.

In accordance with the present invention, nucleic acids having the appropriate sequence homology with an Arabidopsis SDS nucleic acid molecule may be identified by using hybridization and washing conditions of appropriate stringency. For example, hybridizations may be performed, according to the method of Sambrook et al. (1989, supra), using a hybridization solution comprising: 5×SSC, 5×Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° in 1×SSC and 1% SDS, changing the solution every 30 minutes.

One common formula for calculating the stringency conditions required to achieve hybridization between nucleic acid molecules of a specified sequence homology (Sambrook et al., 1989, supra) is:

Tm=81.5EC+16.6 Log[Na+]+0.41(% G+C)−0.63 (% formamide)−600/#bp in duplex

As an illustration of the above formula, using [N+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42 EC. In a preferred embodiment, the hybridization is at 37° C. and the final wash is at 42° C., in a more preferred embodiment the hybridization is at 42° and the final wash is at 50°, and in a most preferred embodiment the hybridization is at 42° C. and final wash is at 65° C., with the above hybridization and wash solutions. Conditions of high stringency include hybridization at 42° C. in the above hybridization solution and a final wash at 65° C. in 0.1×SSC and 0.1% SDS for 10 minutes.

Nucleic acids of the present invention may be maintained as DNA in any convenient cloning vector. In a preferred embodiment, clones are maintained in plasmid cloning/expression vector, such as pBluescript (Stratagene, La Jolla, Calif.), which is propagated in a suitable E. coli host cell.

Arabidopsis SDS nucleic acid molecules of the invention include DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule encoding the protein of the present invention. Such oligonucleotides are useful as probes for detecting Arabidopsis SDS genes or transcripts.

D. Engineering Plants to Alter SDS Activity

While the SDS null mutant of the present invention is a mutant generated by transposable Ds lines, any plant may be transgenically engineered to display a similar phenotype. While the SDS mutant described in the present invention has lost the ability to maintain homolog attachment during meiosis, a transgenic plant can be made that also has a similar loss of the SDS product. This approach is particularly appropriate to plants with high ploidy numbers, including but not limited to wheat.

A synthetic null mutant can be created by a expressing a mutant form of the SDS protein to create a “dominant negative effect”. While not limiting the invention to any one mechanism, this mutant SDS protein will compete with wild-type SDS protein for interacting proteins in a transgenic plant. By over-producing the mutant form of the protein, the signaling pathway used by the wild-type SDS protein can be effectively blocked. Examples of this type of “dominant negative” effect are well known for both insect and vertebrate systems (Radke et al, 1997, Genetics 145:163-171; Kolch et al., 1991, Nature 349:426-428).

A second kind of synthetic null mutant can be created by inhibiting the translation of the SDS mRNA by “post-transcriptional gene silencing”. The SDS gene from the species targeted for down-regulation, or a fragment thereof, may be utilized to control the production of the encoded protein. Full-length antisense molecules or antisense oligonucleotides are used that are targeted to specific regions of the SDS-encoded RNA that are critical for translation. The use of antisense molecules to decrease expression levels of a pre-determined gene is known in the art. Antisense molecules may be provided in situ by transforming plant cells with a DNA construct which, upon transcription, produces the antisense RNA sequences. Such constructs can be designed to produce full-length or partial antisense sequences. This gene silencing effect can be enhanced by transgenically over-producing both sense and antisense RNA of the gene coding sequence so that a high amount of dsRNA is produced (for example see Waterhouse et al., 1998, PNAS 95:13959-13964). In a preferred embodiment, part or all of the SDS coding sequence antisense strand is expressed by a transgene. In a particularly preferred embodiment, hybridizing sense and antisense strands of part or all of the SDS coding sequence are transgenically expressed.

A third type of synthetic null mutant can also be created by the technique of “co-suppression”. Plant cells are transformed with a copy of the endogenous gene targeted for repression. In many cases, this results in the complete repression of the native gene as well as the transgene. In a preferred embodiment, the SDS gene from the plant species of interest is isolated and used to transform cells of that same species.

Transgenic plants can also be created that have enhanced SDS activity. This is an additional way to manipulate meiosis to advantage. This can be accomplished by transforming plant cells with a transgene that expresses part or all of the SDS coding sequence (SEQ ID NO:1), or a sequence that encodes either the SDS protein (SEQ ID NO:2) or a protein functionally similar to it. In a preferred embodiment, the complete SDS coding sequence is transgenically over-expressed. In a particularly preferred embodiment, the coding sequence corresponding to the cyclin domain of SDS is over-expressed.

Transgenic plants with one of the transgenes mentioned above can be generated using standard plant transformation methods known to those skilled in the art. These include, but are not limited to, Agrobacterium vectors, polyethylene glycol treatment of protoplasts, biolistic DNA delivery, UV laser microbeam, gemini virus vectors, calcium phosphate treatment of protoplasts, electroporation of isolated protoplasts, agitation of cell suspensions in solution with microbeads coated with the transforming DNA, agitation of cell suspension in solution with silicon fibers coated with transforming DNA, direct DNA uptake, liposome-mediated DNA uptake, and the like. Such methods have been published in the art. See, e.g., Methods for Plant Molecular Biology (Weissbach & Weissbach, eds., 1988); Methods in Plant Molecular Biology (Schuler & Zielinski, eds., 1989); Plant Molecular Biology Manual (Gelvin, Schilperoort, Verma, eds., 1993); and Methods in Plant Molecular Biology—A Laboratory Manual (Maliga, Klessig, Cashmore, Gruissem & Varner, eds., 1994). The method of transformation depends upon the plant to be transformed. Agrobacterium vectors are often used to transform dicot species. Agrobacterium binary vectors include, but are not limited to, BIN19 and derivatives thereof, the pBI vector series, and binary vectors pGA482 and pGA492. For transformation of monocot species, biolistic bombardment with particles coated with transforming DNA and silicon fibers coated with transforming DNA are often useful for nuclear transformation.

DNA constructs for transforming a selected plant comprise a coding sequence of interest operably linked to appropriate 5′ (e.g., promoters and translational regulatory sequences) and 3′ regulatory sequences (e.g., terminators). In a preferred embodiment, the coding region is placed under a powerful constitutive promoter, such as the Cauliflower Mosaic Virus (CaMV) 35S promoter or the figwort mosaic virus 35S promoter. Other constitutive promoters contemplated for use in the present invention include, but are not limited to: T-DNA mannopine synthetase, nopaline synthase (NOS) and octopine synthase (OCS) promoters.

Transgenic plants expressing a sense or antisense SDS coding sequence under an inducible promoter are also contemplated to be within the scope of the present invention. Inducible plant promoters include the tetracycline repressor/operator controlled promoter, the heat shock gene promoters, stress (e.g., wounding)-induced promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, glucanase genes, chitinase genes, etc.) and dark-inducible gene promoters (e.g., asparagine synthetase gene) to name a few.

Tissue specific and development-specific promoters are also contemplated for use in the present invention. Examples of these included, but are not limited to: the ribulose bisphosphate carboxylase (RuBisCo) small subunit gene promoters or chlorophyll a/b binding protein (CAB) gene promoters for expression in photosynthetic tissue; the various seed storage protein gene promoters for expression in seeds; and the root-specific glutamine synthetase gene promoters where expression in roots is desired. The SDS promoter as set forth in SEQ ID NO:3 of the present invention is the first known meiosis specific promoter. The 5′ end of SEQ ID NO:3 has an 110 base pair overlap with a putative gene. The 3′ end of SEQ ID NO:3 has approximately 120 base pair overlap with the SDS transcribed region. Confirmation that SEQ ID NO:3 is the SDS promoter was made by forming a fusion between the SDS promoter with a GUS reporter gene and examining meiosis-specific expression of the GUS reporter.

The coding region is also operably linked to an appropriate 3′ regulatory sequence. In a preferred embodiment, the nopaline synthetase polyadenylation region (NOS) is used. Other useful 3′ regulatory regions include, but are not limited to the octopine (OCS) polyadenylation region.

Using an Agrobacterium binary vector system for transformation, the selected coding region, under control of a constitutive or inducible promoter as described above, is linked to a nuclear drug resistance marker, such as kanamycin resistance. Other useful selectable marker systems include, but are not limited to: other genes involved in meosis or the regulation of meiosis.

Plants are transformed and thereafter screened for one or more properties, including the lack of SDS protein, SDS mRNA, or abnormal meiosis—particularly failure to maintain homolog attachment. It should be recognized that the amount of expression, as well as the tissue-specific pattern of expression of the transgenes in transformed plants can vary depending on the position of their insertion into the nuclear genome. Such positional effects are well known in the art. For this reason, several nuclear transformants should be regenerated and tested for expression of the transgene.

Transgenic plants that exhibit one or more of the aforementioned desirable phenotypes can be used for plant breeding, or directly in agricultural or horticultural applications. Plants containing one transgene may also be crossed with plants containing a complementary transgene in order to produce plants with enhanced or combined phenotypes. Further, plants could be generated which are SDS deficient, resulting in failure to produce pollen and/or sterility.

E. In Vivo Synthesis of the SDS Protein

The availability of amino acid sequence information, such as the full length sequence in SEQ ID NO: 2, enables the preparation of a synthetic gene that can be used to synthesize the Arabidopsis SDS protein in standard in vivo expression systems, or to transform different plant species. The sequence encoding Arabidopsis SDS from isolated native nucleic acid molecules can be utilized. Alternately, an isolated nucleic acid that encodes the amino acid sequences of the invention can be prepared by oligonucleotide synthesis. Codon usage tables can be used to design a synthetic sequence that encodes the protein of the invention. In a preferred embodiment, the codon usage table has been derived from the organism in which the synthetic nucleic acid will be expressed. For example, the codon usage for pea (Pisum sativum) would be used to design an expression DNA construct to produce the Arabidopsis SDS in pea. Synthetic nucleic acid molecules may be prepared by the phosphoramadite method employed in the Applied Biosystems 38A DNA Synthesizer or similar devices, and thereafter may be cloned and amplified in an appropriate vector.

The availability of nucleic acids molecules encoding the Arabidopsis SDS protein enables production of the protein using in vivo expression methods known in the art. According to a preferred embodiment, the protein may be produced by expression in a suitable expression system. The SDS protein of the present invention may also be prepared by in vitro transcription and translation of either native or synthetic nucleic acid sequences that encode the proteins of the present invention. While in vitro transcription/translation is not the method of choice for preparing large quantities of the protein, it is ideal for preparing small amounts of native or mutant proteins for research purposes, particularly since in vitro methods allow the incorporation of radioactive nucleotides such as 35S-labeled methionine. The SDS proteins of the present invention may be prepared by various synthetic methods of peptide synthesis via condensation of one or more amino acid residues, in accordance with conventional peptide synthesis methods. The SDS produced by native cells or by gene expression in a recombinant procaryotic or eukaryotic system may be purified according to methods known in the art.

F. Antibodies Immunospecific to SDS

The present invention also provides antibodies that are immunologically specific to the SDS protein of the invention. Polyclonal antibodies may be prepared according to standard methods. In a preferred embodiment, monoclonal antibodies are prepared, which immunologically specific to various epitopes of the protein. Monoclonal antibodies may be prepared according to general methods of Kohler and Milstein, following standard protocols. Polyclonal or monoclonal antibodies that are immunologically specific to the Arabidopsis SDS can be utilized for identifying and purifying SDS from Arabidopsis and other species. For example, antibodies may be utilized for affinity separation of proteins for which they are immunologically specific or to quantify the protein. Antibodies may also be used to immunoprecipitate proteins from a sample containing a mixture of proteins and other biological molecules.

IV. Use of SDS Nucleic Acids, SDS Proteins and Antibodies, and Transgenic Plants.

A. Uses of SDS nucleic acids.

SDS nucleic acids may be used for a variety of purposes in accordance with the present invention. DNA, RNA, or fragments thereof may be used as probes to detect the presence and/or expression of SDS genes. Methods in which SDS nucleic acids may be utilized as probes for such assays include, but are not limited to: (1) in situ hybridization; (2) Southern hybridization (3) Northern hybridization; and (4) assorted amplification reactions such as polymerase chain reactions (PCR).

The SDS nucleic acids of the invention may also be utilized as probes to identify related genes from other plant species. As is well known in the art, hybridization stringencies may be adjusted to allow hybridization of nucleic acid probes with complementary sequences of varying degrees of homology. As described above, SDS nucleic acids may be used to advantage to produce large quantities of substantially pure SDS, or selected portions thereof. The SDS nucleic acids can be used to identify and isolate further members of the meiosis regulatory pathway in vivo. A yeast two hybrid system can be used to identify proteins that physically interact with the SDS protein, as well as isolate their nucleic acids. In this system, the sequence encoding the protein of interest is operably linked to the sequence encoding half of a activator protein. This construct is used to transform a yeast cell library which has been transformed with DNA constructs that contain the coding sequence for the other half of the activator protein operably linked to a random coding sequence from the organism of interest. When the protein made by the random coding sequence from the library interacts with the protein of interest, the two halves of the activator protein are physically associated and form a functional unit that activates the reporter gene. In accordance with the present invention, all or part of the Arabidopsis SDS coding sequence may be operably linked to the coding sequence of the first half of the activator, and the library of random coding sequences may be constructed with cDNA from Arabidopsis and operably linked to the coding sequence of the second half of the activator protein. Several activator protein/reporter genes are customarily used in the yeast two hybrid system. In a preferred embodiment, the bacterial repressor LexA DNA-binding domain and the Gal4 transcription activation domain fusion proteins associate to activate the LacZ reporter gene (see Clark et al., 1998, PNAS 95:5401-5406). Kits for the two hybrid system are also commercially available from Clontech, Palo Alto Calif., among others.

SDS Nucleic acids and proteins of the invention are also useful for plant breeding purposes. Plants which are sds mutant are typically male sterile. Such plants are valuable for generation of hybrid seeds and hybrid plant varieties. These male sterile varieties would not generate pollen, thus being valuable for containment purposes in cross-breeding. Further, ornamental flowering plants, such as pear, cherry, crabapple, and the like which are mutant in the SDS gene are defective in pollen production. In other words, such sds mutant plants flower but do not pollen, thus conferring a practical and commercial benefit on such varieties. Finally, SDS mutant plants are useful in apomixis, wherein seed production bypasses meiosis, effectively resulting in generation of identical (i.e., cloned) progeny seeds.

B. Uses of SDS proteins and antibodies:

The SDS proteins of the present invention can be used to identify molecules with binding affinity for SDS, which are likely to be novel participants in the meiotic regulatory pathway. In these assays, the known protein is allowed to form a physical interaction with the unknown binding molecule(s), often in a heterogenous solution of proteins. The known protein in complex with associated molecules is then isolated, and the nature of the associated protein(s) and/or other molecules is determined.

Antibodies that are immunologically-specific for SDS may be utilized in affinity chromatography to isolate the SDS protein, to quantify the SDS protein utilizing techniques such as western blotting and ELISA, or to immuno-precipitate SDS from a sample containing a mixture of proteins and other biological materials. The immuno-precipitation of SDS is particularly advantageous when utilized to isolate affinity binding complexes of SDS, as described above.

### EXAMPLES

### Example 1

Generation and Identification of the Sds Mutant

To generate insertional mutations in Arabidopsis, a two element system was established using the maize transposable elements Ac and Ds. This modified Ac/Ds system was previously described by Sundaresan et al (1995) Genes & Dev., 9:

1797-1810, and confers efficient transposition in Arabidopsis. Approximately 2000 independent Ds insertional lines with Ds elements were scattered throughout the genome. These were screened for visible phenotypes during development, with a particular emphasis on flower and pollen development. Several candidate male sterile mutants were found; one of them showed a defect in pollen size and was further characterized.

Genetic studies indicated that the mutation having a defect in pollen size was a recessive nuclear mutation, and that the mutant was female fertile with no difference from the normal plant. Normal plant male meiosis produces four equal-sized microspores enclosed in a structure known as a tetrad. The microspores are subsequently released from the tetrad and develop into mature pollen grains. Previous studies have shown that when male meiosis is abnormal due to a mutation, the number and size of microspores are often abnormal. In addition, the size variation can be observed during later pollen development. Therefore, the initial observation that the new mutant also produced microspores and pollen grains of variable sizes suggested that it might be defective in meiosis. Examination using light microscopy confirmed that male meiosis in this mutant was defective, producing tetrads with 4-8 microspores having uneven sizes.

To further examine the defects in the mutant, a detailed analysis was undertaken using fluorescence light microscopy following the procedure described in Ross et al., (1996) Chromosome Res., 4: 507-516; Ross et al., (1997) Chromosome Res., 5: 551-559. It was observed that the mutant was abnormal in the pairing of homologous chromosomes during meiotic prophase I. During normal Arabidopsis meiosis I prophase, homologous chromosomes pair to form five pairs. However, in the mutant, this pairing was defective, as was evidenced by the observation of individual unpaired chromosomes. As a result, chromosome distribution was uneven during the subsequent anaphase I and telophase I. Specifically, instead of the normal distribution of 5 chromosomes to each of two poles at the end of meiosis I, the mutant exhibited segregation of anywhere between 2-8 chromosomes at one pole. Often, one or more chromosomes remained near the equatorial plane in between the two poles. Therefore, at the end of meiosis I, the mutant had 2-4 groups of chromosomes. During meiosis II, each chromosome then divided into two chromatids that were able to separate normally. The number of spores was then twice the number of chromosomal groups at the end of meiosis I and the size of individual microspores correlated with the number of chromosomes(s) in a group. These defects are sufficient to explain the abnormal number and size of the microspores that formed following meiosis II. Among more than 100 meioses examined, none were found to show normal chromosome segregation. Therefore, the mutant defect in male meiosis was determined to be the cause of male sterility.

Normal spindle function is critical for proper chromosome distribution during meiosis. The fact that in the mutant some chromosomes were able to move the two poles during meiosis I suggest that the meiosis I spindle was functioning. In addition, the spindles in meiosis II functioned properly to separate of all sister chromatids. Therefore, the mutant defect was not the result of a defective spindle. It is possible that the defect in pairing of homologous chromosomes is the primary defect. Because of the defect in homologous chromosome pairing is the earliest defect observed, and because in normal meiosis homologous chromosome pairing is a highly regulated event, similar to a choreographed dance of couples, the mutant was named, “solo dancers” (sds) to illustrate the phenotype.

### Example 2

Isolation of the SDS Gene

The sds mutant was identified among Ds transposon insertional lines. A single Ds element was detected in the mutant genomic DNA by Southern DNA hybridizations. The genomic DNA sequence flanking the Ds element using the TAIL PCR procedure as previously described (Liu et al., 1995 Plant J., 8: 457-463). Using primers designed on the basis of the plant sequences, PCR amplification was conducted. A fragment of expected size was amplified from the wild-type DNA but not the mutant DNA.

The sds mutant carries a co-segregating Ds insertion. If this Ds is inserted in the SDS gene, excision of the Ds could produce normal-appearing revertant sectors. A screen was conducted among sds mutant plants carrying an Ac. Nine of thirteen sds plants produced phenotypically normal revertant sectors such as a branch. The flowers of one large sector produced normal pollen thus ruling out the possibility that the seeds of the revertant sector were due to contaminating pollen. Furthermore, seeds of three revertant sectors were planted, and each segregated for mutant plants, indicating that the sector was heterozygous for the SDS gene and consistent with the sector being a revertant. Therefore, the sds mutant was most likely caused by a Ds insertion.

To isolate the SDS gene, approximately 0.8 kb and 0.6 kb fragments were obtained from the 5′ and 3′ ends of the Ds element, respectively, using the TAIL-PCR procedure. Sequence analysis indicates that these fragments share the same 8 basepairs immediately adjacent to the Ds sequences, consistent with the 8 base pair duplication characteristic of Ds insertions. Therefore, these two TAIL-PCR fragments are indeed most likely derived from either side of the same Ds element, and should be portions of the SDS gene.

As described above, nine revertant sectors were isolated. Using primers matching genomic sequences flanking the Ds element, we amplified a PCR fragment of the expected size from wild-type and revertant genomic DNAs, but not from mutant DNAs. The sequences of 7 revertants near the Ds insertion site was determined. Four of the revertants have wild-type sequences, while the other three have 6 or 9 bp insertions (Ds excision footprints) which would not disrupt an open reading frame. Because the revertant sequences restored gene function and could not have been from a wild-type copy, these results confirm the identification of the genomic insertion that is responsible for the sds mutant phenotype, and that the insertion disrupts a protein coding region.

Using the SDS genomic sequence as a probe, a 2.4 kb cDNA clone was isolated after screening about 1 million plaques of a cDNA library made from mRNAs of young flowers containing meiotic cells. The SDS sequence also matches to a sequenced BAC clone from chromosome 1. Sequence of the SDS cDNA (SEQ ID NO:1) predicts an open reading frame of 578 amino acid residues (SEQ ID NO:2). The C-terminal one third of the predicted SDS protein is similar to the cyclin box of several known Arabidopsis cyclins. Like other cyclins, the N-terminal region is not conserved. Because the levels of amino acid sequence identity between SDS and known cyclins are close to the levels between different types of known Arabidopsis cyclins, and less than the levels between members of the same types (greater than 70% identity), we assert that SDS is a new type of cyclin. Known yeast cyclins have been found to be important for both mitosis and meiosis. A cyclin specifically required for homolog attachment during meiosis I has not been described. In addition, SDS is different from other Arabidopsis meiotic genes cloned to date, including SYN1, AtDMC1, and ASK1.

To determine whether SDS has any closely related genes, Southern analysis was conducted. The BAC clone containing the SDS gene or total Arabidopsis genomic DNA was digested with EcoRI (R), HindIII (H), or XhoI (X) and hybridized with the SDS cDNA as a probe at a high stringency. Only predicted bands were observed. However, when the same genomic DNAs were probed under moderate stringency (55° C. washes with 0.5×SSC), additional band(s) were detected in all three digests, suggesting the presence of another gene with a high degree of sequence similarity. The closest known genes (encoding AtcycB2-1 and AtcycB2-2) are not similar enough to be detectable by Southern hybridization.

### Example 3

Expression of SDS

Northern hybridization experiments indicated that the level of SDS mRNA is low in Arabidopsis inflorescences, and is not detectable in other organs. RT-PCR using SDS-specific primers and mRNA from roots, leaves, floral stems, inflorescence, open flowers, and seedpods indicate that SDS expression is only detectable in inflorescences with immature floral buds. RNA in situ hybridization was performed with sections of inflorescences. Results show that SDS is not expressed in the inflorescence meristem or very early floral primordia. It is still not detectable at stage 8 developing flower, when anther differentiation has begun but meiosis has not occurred. At about stage 9, or the time of meiosis, a strong signal was observed that was restricted to the microspore mother cells within the anther, but not in other tissues of the anther, nor in other floral organs. At stage 11 during pollen development, SDS expression is again not detectable. Therefore, SDS is expressed in a highly specific manner both spatially and temporally in the male.

### Example 3

Yeast Two-Hybrid Selections for SDS-Interacting Proteins.

In order to identify putative SDS-interacting proteins, a yeast two-hybrid selection was performed using a yeast strain generated by James et al. Genetics, 144: 1425-1436. This yeast strain has three reporter genes (HIS3, ADE2, and lacZ) with different GAL4-responsive promoters and was shown to be much less prone to the problems of false positives. We have previously generated cDNA library from mRNAs isolated from immature floral buds containing meiotic cells. The SDS cDNA was isolated from this library. We recently generated a bait construct with the entire SDS protein coding sequence fused to the yeast GAL4 DNA-binding domain. The bait construct allowed minimal growth on media lacking histidine, which is eliminated by using 10 mM 3-amino-triazol, an his analog. Approximately 500,000 transformants carrying the bait and the cDNA library plasmid were generated. Among them, more than 100 were putative His+ transformants.

The present invention is not limited to the embodiments described and exemplified above, but is capable of variation and modification without departure from the scope of the appended claims.

What is claimed:

1. An isolated nucleic acid molecule, comprising a gene located on Arabidopsis thaliana chromosome 1, the disruption of which is associated with a failure to maintain homolog attachment during meiotic prophase I.

2. The nucleic acid molecule of claim 1, which encodes a protein having a cyclin domain.

3. The nucleic acid molecule of claim 2, wherein the gene is composed of exons that form an open reading frame having a sequence that encodes a polypeptide approximately 578 amino acids in length.

4. A cDNA molecule comprising the exons of the nucleic acid of claim 3.

5. The nucleic acid of molecule of claim 3, wherein the open reading frame encodes an amino acid sequence at least 70% identical to the cyclin domain of SEQ ID NO:2.

6. The nucleic acid molecule of claim 3, wherein the open reading frame encodes an amino acid sequence which is at least 50% identical to SEQ ID NO:2 over the entire length of SEQ ID NO:2.

7. The nucleic acid molecule of claim 6, wherein the open reading frame encodes SEQ ID NO:2.

8. The nucleic acid molecule of claim 6, which comprises an open reading frame having the sequence set forth in SEQ ID NO:1.

9. An oligonucleotide between about 15 and 100 nucleotides in length, which specifically hybridizes with either strand of the nucleic acid molecule of claim 1.

10. A polypeptide produced by expression of the nucleic acid molecule of claim 1.

11. Antibodies immunologically-specific for the polypeptide of claim 9.

12. A vector for transforming a plant cell, comprising the nucleic acid molecule of claim 1.

13. A transformed plant cell comprising the vector of claim 12.

14. An isolated nucleic acid molecule comprising an open reading frame of a gene located on Arabidopsis chromosome 1, the open reading frame having a sequence selected from the group consisting of:
a) SEQ ID NO:1;
b) a sequence that is at least 80% identical to SEQ ID NO: 1;
c) a sequence encoding a polypeptide having SEQ ID NO:2;
d) a sequence encoding a polypeptide having a at least 50% identity to SEQ ID NO:2;
e) a sequence encoding a polypeptide having at least 70% identity to the cyclin domain of SEQ ID NO:2; and
f) a nucleotide sequence that hybridizes with SEQ ID NO:1 under stringent conditions,
wherein stringent conditions are
hybridizing for at least 6 hours at 37° C. in 5×SSC, 5×Denhardt's reagent, 1.0% SDS, 100 μg/ml denatured fragmented salmon sperm DNA, 0.05% sodium pyrophosphate; and
washing once for 5 minutes at room temperature in 2×SSC and 1% SDS, once for 15 minutes at room temperature in 2×SSC and 0.1% SDS, once for 30 minutes at 37° C. in 1×SSC and 1% SDS and four times for 30 minutes each at 42° C. in 1×SSC and 1% SDS.

15. A polypeptide, produced by the expression of the isolated nucleic acid molecule of claim 14.

16. Antibodies immunologically specific for the polypeptide of claim 15.

17. A vector for transforming a plant cell, comprising the nucleic acid molecule of claim 14.

18. A transformed plant cell comprising the vector of claim 17.

19. A plant comprising a mutation in an SDS gene, wherein said mutation confers an inability to maintain homolog attachment during meiosis.

20. A plant gene promoter comprising a nucleic acid sequence which when operatively linked to a cDNA sequence, confers meiosis-specific expression on said cDNA sequence.

21. An isolated nucleic acid comprising an SDS promoter, wherein said promoter comprises the sequence set forth in SEQ ID NO:3.

22. An isolated nucleic acid comprising a genomic SDS sequence, wherein said sequence is at least 70% identical to that of SEQ ID NO:4, over the entire length of SEQ ID NO:4.

23. The isolated nucleic acid of claim 22, wherein said sequence comprises the polynucleotide sequence of SEQ ID NO:4.

24. A plant cell comprising a mutation in an SDS gene, wherein such mutation confers onto said plant cell at least one of the phenotypes of sterility and inability to produce pollen.


SEQUENCE LISTING (7 sequences)

SEQ ID NO: 1 - DNA, 2144 bp, Arabidopsis thaliana
actgcatcagcccactctctagtctctgactaacgaacttccattttcaaaattcgaatttctaatttctagtttcaagctttcgtacggagaaaaaatgaaggagatcgcgatgaggaattcaaagcgcaagcctgagccgacgccgttcgccgggaagaagctccggtcgacgcgattacgccggaagagagcacagatctctcccgttcttgttcaatcacctctctggagcaaacaaatcggagtctctgctgcttctgtcgattcctgctccgatttgctagctgatgacaacgtttcctgtggttcgagcagagtcgagaagagctcgaatccgaagaagactctaattgaagaggtagaagtttctaaacctggttataatgtgaaggagacgattggtgattcgaaatttcgaaggattacgaggtcttactctaagctacacaaggagaaggagggagatgagatcgaagtaagcgaatcgtcttgtgttgattcgaattctggtgctggattaaggagattgaatgtgaagggaaataaaattaacgacaacgatgagatctctttctcacgatccgatgtgaccttcgccggacatgtctccaacagccggagtttgaatttcgaatcggagaataaggagagcgacgtcgtttctgtcatatctggagttgagtactgttccaagttcgggagcgttaccggaggagctgataacgaagaaattgaaatctccaagccgagcagcttcgtggaagctgattcctctcttggatcggccaaggaattgaagccggagcttgagatagtcggatgcgtctctgatctcgcttgctctgagaaattctcggaagaggtttcggattctctcgatgatgagtcatctgagcaacgttcagagatatattcacagtattccgacttcgattactcggattacactccgtccatcttcttcgactctggcagcgaattctctgagaaatcttcctctgattctcctatttcacattctcgctctctgtacctccagttcaaggaacagttctgtagatccacgattcccaacgattttggatcttcttgcgaggaagaaattcactctgaattgctaaggtttgatgatgaggaggtggaagagagctatctaaggctgagggaaagagaaagaagtcatgcatatatgcgggactgtgctaaggcatactgctccaggatggacaatactggtctcatccctcgtctacgctccatcatggttcaatggattgtaaagcaatgttctgacatggggcttcagcaagagacattgtttctaggagttggtctgttggatcgattcctgagcaaaggatcattcaaaagcgaaaggactctaatactagtcgggattgcgagtcttactctggccaccagaattgaagaaaatcaaccttacaacagcatccggaaaaggaacttcaccattcagaacctaagatatagccggcatgaagtggtggcaatggagtggctggttcaagaagtcctcaacttcaaatgcttcacacccacaatcttcaacttcttgtggttctacttaaaagctgctcgagccaatccagaagttgaaaggaaagccaaatccttggctgttacctcactatccgaccaaactcaactctgtttttggccctcaactgtagcagctgcactcgtggttctcgcctgcatcgaacacaacaaaatctctgcataccaacgagtcataaaggtccatgttagaacaacagataacgagttgcctgaatgcgttaagagtctggactggttgcttgggcagtaagcaatcaaaaagaacaaaaaccctaaaaccaggacacagtatactccgataccaacacacaggttatcattactatttacaaaaacaaacacaaggtaagtaataagaactcctctacagatttatatacttaatcgagctggacttaattagctcttagtataccaattattagtgccaccatttgtgtcgctcatacacatttatttcttattttccctaattcattagactctcatattcttaaaaagaatatttccttgtttgaaaaaaaaaaaaaaaaaaaaaaaaaaaaaaaaaaaaaaaaaaa

SEQ ID NO: 2 - protein, 578 aa, Arabidopsis thaliana
MKEIAMRNSKRKPEPTPFAGKKLRSTRLRRKRAQISPVLVQSPLWSKQIGVSAASVDSCSDLLADDNVSCGSSRVEKSSNPKKTLIEEVEVSKPGYNVKETIGDSKFRRITRSYSKLHKEKEGDEIEVSESSCVDSNSGAGLRRLNVKGNKINDNDEISFSRSDVTFAGHVSNSRSLNFESENKESDVVSVISGVEYCSKFGSVTGGADNEEIEISKPSSFVEADSSLGSAKELKPELEIVGCVSDLACSEKFSEEVSDSLDDESSEQRSEIYSQYSDFDYSDYTPSIFFDSGSEFSEKSSSDSPISHSRSLYLQFKEQFCRSTIPNDFGSSCEEEIHSELLRFDDEEVEESYLRLRERERSHAYMRDCAKAYCSRMDNTGLIPRLRSIMVQWIVKQCSDMGLQQETLFLGVGLLDRFLSKGSFKSERTLILVGIASLTLATRIEENQPYNSIRKRNFTIQNLRYSRHEVVAMEWLVQEVLNFKCFTPTIFNFLWFYLKAARANPEVERKAKSLAVTSLSDQTQLCFWPSTVAAALVVLACIEHNKISAYQRVIKVHVRTTDNELPECVKSLDWLLGQ

SEQ ID NO: 3 - DNA, 3018 bp, Arabidopsis thaliana
gtcgaccagagtttgaccaatgactaatgttatcgtatgattcaattattttttgtacagtaatgtctcgtagaccgacaacaagaccagaaagtaatcttaaaaccctagcttcacacttagatatgtttctatacatgtgtgtatatatacacacatacttgtagagcatgtgagacagtgaaatttactagtgttaaacatggatgtgaagaaagatgagaacagtatcttagaaaacatgaaacaagagattaatcatagtctaaaagaagaagcacaagaagaagaagagattctaaagaagagaatctcaagccaccctttgtatgggcttcttcttcactcacatctcaattgtttaaaggtacccctcttaatgcttccctctctctcttttttattaaagtgatgtatgagtataaatgtttatctcttatgtatttggacccaaccacgaccaggtgtgttccggcgactttgactcaccggagatcatgaacacggctgatgatcttgccctatccaaactctctctccaccctgactcttcctccgaagctacctcttcagaacttgatcaattcatggttcttttttttttttctccctgccaaaacatatttacacaacaaaaaacaacttttcacgttcttttattttttcctttgcaaattaatttgactttcaagtattctaagtttattttgccaagaaaaaaacagtagcacttatacttgtaaattgattcacataataggaagcgtattgctcgactctacgggagctcaaggaagcaatggagaagcctcttaccgaaacgcatcgttttgtggatgcggtgtacactcagctaaacgacatcgttatgtcatcacccccttaaaaaagggtaacatgaacaactgttcggtgctactatgtcaatgcattttgccaaattactactcagtctactcacgatttattgtactgcgtttacgtaacgcgtttgtatgatcgtttattggtaaccgtaatttatggcatgccctcctgcttttttatttaagaaaaataaaactaattatattgtaaatattgcattgatcatttagtcacactctttagaaaacaacagtaaaatttaaatataaaaacaacactagcttccatgattatttttcataaccatttataattgcgtcatcttgtaagttgtaacgcattgcctttcttactatgtaacggttgttgcatatttttgtgtacataaatttatacacaaagataaaaagtgactaagcttaaaatatccttgaaaaagcctttgggtcattaacatggtgtaagactacaggcgcattcagcaattggagttccgattctattacagtaagagggaacagaaccgtaataatcgcgacacatttgttcgcatttgttagcatcgcatggaaccattggccagaaaacggggcaagtttgttccatcattctcgtctctctcgcacctttaaacaaacatcagaaaatttgtgacattaattaacaggatttggcttcttataaagataagattaaaactactatttaaaagataatctgtacctgaggctgaaacgatgaagatggtcatgataagaacagcgaaatttatgaggtttctcatggttttatgtttttttttttcttaacaaagacgtaaacttgaatcgttttatatgcgaaattgacagagaaaaccggaaaagataggatctccttttctttctttcttttagtgaaatagatgataaacttgtttctgctaaaagaggtgtttattttggaaattatgaattttctggtcaatgtgatcttagaattttaaataggctggattttgtgacctgattccgtgtcttatatctgtatttactatatttagatgattctctgataactgatgttttaaaaagaagataattttgataaagaagtgattacgaactttccaacattaaaagtttagagtttatttgattttatatctaatcttggtttatatgtttttgatggggtttactaattatattataccattcaagttgaaatatatacaagttttttttgttttatccctaaattctctaatgtgatatatataatatataatttggatcggattcaaccaaaccatgaacgagatttacattttgccgttttccgaaatgttttgggcttcgtaaagaactaaaggtgatatttagatattgggtatactatttgttgtattgggcttaaaagtttacttttttggcccaaaattaatcaactaaaataagatcaccaatggaaaaagaaacaaaaaaaccagtaaaacatatgcagaaaatgtaaatttacagggcctaatataatctgcttgaccatgccattgcgacataacaaatgttacacaagtagtgtacctataaagtagtgtacctataatatattaacagtgatcaatttcagtgtataaaaaaagtcttcttaaatcatcttttaattccaacaatatgacattcacaaacttatctatgatttttttaaaaaaaaattcacacgtgtgctcaatttatgtttcttttagttcttccacgtgatttgatgcaagaaaaatgattagactgtatgttaaaaagcatactagagaaattaattataaaacatcaatcagttgaagtaattatcaaaaccgcatgcttttttagctaaatctgtgattgtactgacgcagatgcataaattcaaacgcaaacgctgatctctacattagccaaacaagaatagcgtccaaatttacgactggtttcacgtgcaccaaaccgtagggtataatatctctctctcactctccaacatccccactcttcccaagaaacttctataactgcatcagcccactctctagtctctgactaacgaacttccattttcaaaattcgaatttctaatttctagtttcaagctttcgtacggagaaaaaatgaaggagatcgcgatgaggaattc

SEQ ID NO: 4 - DNA, 3970 bp, Arabidopsis thaliana
ttgaccatgccattgcgacataacaaatgttacacaagtagtgtacctataaagtagtgtacctataatatattaacagtgatcaatttcagtgtataaaaaaagtcttcttaaatcatcttttaattccaacaatatgacattcacaaacttatctatgatttttttaaaaaaaaattcacacgtgtgctcaatttatgtttcttttagttcttccacgtgatttgatgcaagaaaaatgattagactgtatgttaaaaagcatactagagaaattaattataaaacatcaatcagttgaagtaattatcaaaaccgcatgcttttttagctaaatctgtgattgtactgacgcagatgcataaattcaaacgcaaacgctgatctctacattagccaaacaagaatagcgtccaaatttacgactggtttcacgtgcaccaaaccgtagggtataatatctctctctcactctccaacatccccactcttcccaagaaacttctataactgcatcagcccactctctagtctctgactaacgaacttccattttcaaaattcgaatttctaatttctagtttcaagctttcgtacggagaaaaaatgaaggagatcgcgatgaggaattcaaagcgcaagcctgagccgacgccgttcgccgggaagaagctccggtcgacgcgattacgccggaagagagcacagatctctcccgttcttgttcaatcacctctctggagcaaacaaatcggagtctctgctgcttctgtcgattcctgctccgatttgctagctgatgacaacgtttcctgtggttcgagcagagtcgagaagagctcgaatccgaagaagactctaattgaagaggtagaagtttctaaacctggttataatgtgaaggagacgattggtgattcgaaatttcgaaggattacgaggtcttactctaagctacacaaggagaaggagggagatgagatcgaagtaagcgaatcgtcttgtgttgattcgaattctggtgctggattaaggagattgaatgtgaagggaaataaaattaacgacaacgatgagatctctttctcacgatccgatgtgaccttcgccggacatgtctccaacagccggagtttgaatttcgaatcggagaataaggagagcgacgtcgtttctgtcatatctggagttgagtactgttccaagttcgggagcgttaccggaggagctgataacgaagaaattgaaatctccaagccgagcagcttcgtggaagctgattcctctcttggatcggccaaggaattgaagccggagcttgagatagtcggatgcgtctctgatctcgcttgctctgagaaattctcggaagaggtttcggattctctcgatgatgagtcatctgagcaacgttcagagatatattcacagtattccgacttcgattactcggattacactccgtccatcttcttcgactctggcagcgaattctctgagaaatcttcctctgattctcctatttcacattctcgctctctgtacctccagttcaaggaacagttctgtagatccacgattcccaacgattttggatcttcttgcgaggaagaaattcactctgaagtaagtggtataatgatttcatatctcttggaataattgctagtggttagagattgaagatgtatgtggttatatggttgaaatttcattcgattactagtctatttttgatatgagacttgttctgctctgtgtttgattctgaaattttgttctggaatgaatcttaagtatacattttcgttttagttgctaaggtttgatgatgaggaggtggaagagagctatctaaggctgagggaaagagaaagaagtcatgcatatatgcgggactgtgctaaggcatactgctccaggatggacaatactggtctcatccctcgtctacgctccatcatggttcaatggattgtaaaggtgaattttaactttctgttcaaatgcatttagttacatatacattgatctctgaatgttgaagctcagaaatatgtatcagtagcagaagattatgaagtaaatgaatatttggagatcctgttcctggttttaagaatgttttagcctaaggaaatctatagcttactttggaatcttttaaggtttatgtatcagtcagctatgatattctttgttgctgattgtctgctccctgattacaagcagcaatgttctgacatggggcttcagcaagagacattgtttctaggagttggtctgttggatcgattcctgagcaaaggatcattcaaaagcgaaaggactctaatactagtcgggattgcgagtcttactctggccaccagaattgaagaaaatcaaccttacaacaggtaccaaccatattccatcttcatgattctgacttccaatgttcattagaaaagtgttctgagtaggaaaaagattaggaccattacaagaaactgagtattacgcttaaccaaatcaaggactaataatggtctaatacaaacccttatggttcaatgaattggcatttcatgtgggtatcgaatattggattatgtttctcaaaaacactctttactggaaagaaccttccacaatacacaggaatagttcaattttcttcaactgctcacctgatacttgctctttttaactagcatccggaaaaggaacttcaccattcagaacctaagatatagccggcatgaagtggtggcaatggagtggctggttcaagaagtcctcaacttcaaatgcttcacacccacaatcttcaacttcttgtggtaaaacctctctgactatatattttcatgttccaagacacattatccacacagaaagatacatatgactatcatttatacatgtcaggttctacttaaaagctgctcgagccaatccagaagttgaaaggaaagccaaatccttggctgttacctcactatccgaccaaactcaactctgtttttggccctcaactgtagcagctgcactcgtggttctcgcctgcatcgaacacaacaaaatctctgcataccaacgagtcataaaggtatcatcagtcccttcaataacactttaataccttttagtatcgagaatatacaagaatcttcacaatcccaaaacctctctttctctccaggtccatgttagaacaacagataacgagttgcctgaatgcgttaaggtgttttcagtaacactctcattatatacaaatctcatttttaccactaaacgtaaggtaagtgactgttttcacatttttgttccctatacaacagagtctggactggttgcttgggcagtaagcaatcaaaaagaacaaaaaccctaaaaccaggacacagtatactccgataccaacacacaggttatcattactatttacaaaaacaaacacaaggtaagtaataagaactcctctacagatttatatacttaatcgagctggacttaattagctcttagtataccaattattagtgccaccatttgtgtcgctcatacacatttatttcttattttccctaattcattagactctcatattcttaaaaagaatatttccttgtttgatatttcctctttattacgtatgaaaggttttcaatttttctaatcttcactgtttctgatctcaaatatgaaaaagcttatcaaatccagcttaaaaagagagtgtagtcgaatttaactaaagatttaaaaatggtaatgtttttcttcacacacgctataggatgcattctcgacggtaatcaaattaaacagtttgtaaaatggttaaagcaaaacatgcttgtaaactagagatttttttttcgatcaaaaattagagatttatgtatgtaacaattatagaataaatactagcatatatgtatgtacgtacataaaagtcagtctaattctctaataaata

SEQ ID NO: 5 - protein, 145 aa, Arabidopsis thaliana
MDNTGLIPRLRSIMVQWIVKQCSDMGLQQETLFLGVGLLDRFLSKGSFKSERTLILVGIASLTLATRIEENQPYNSIRKRNFTIQNLRYSRHEVVAMEWLVQEVLNFKCFTPTIFNFLWFYLKAARANPEVERKAKSLAVTSLSD

SEQ ID NO: 6 - protein, 143 aa, Arabidopsis thaliana
MAQQFDISDKMRAILIDWLIEVHDKFELMNETLFLTVNLIDRFLSKQAVARKKLQLVGLVALLLACKYEEVSVPIVEDLVVISDKAYTRTDVLEMEKIMLSTLQFNMSLPTQYPFLKRFLKAAQSDKKLEILASFLIELALVD

SEQ ID NO: 7 - protein, 142 aa, Arabidopsis thaliana
MQQIDLNEKMRAILIDWLIEVHDKFDLMNETLFLTVNLIDRFLSKQNVMRKKLQLVGLVALLLACKYEEVSVPVVEDLVLISDKAYTRNDVLEMEKTMLSTLQFNISLPTQYPFLKRFLKAAQADKKCEVLASFLIELALVE